(19) United States
(12) Patent Application Publication
(10) Pub. No.: US 2006/0173300 A1
(43) Pub. Date: Aug. 3, 2006

(54) Open structure sizing device

(75) Inventors: John C. Oslund, Blaine, MN (US); Gary A. Thill, Vadnais Heights, MN (US); Xiaoping Gu, Maplewood, MN (US)

Correspondence Address: NIKOLAI & MERSEREAU, P.A., 900 SECOND AVENUE SOUTH, SUITE 820, MINNEAPOLIS, MN, 55402, US

(73) Assignee: AGA Medical Corp., Golden Valley, MN (US)

(21) Appl. No.: 11/033,233

(22) Filed: Jan. 11, 2005

(51) Int. Cl. A61M 25/00; A61B 6/00

(52) U.S. Cl. 600/435; 600/433

(57) ABSTRACT

A system and method for sizing and imaging of cardiovascular defects generally are disclosed capable of producing model images or forms that mimic the shape of defects such as openings or breaches in cardiac septum walls, so that remedial devices of the correct size and shape can be administered to occlude defects. The system uses a three-dimensional open wire imaging structure which is flexible, easy to position and does not occlude blood flow and when expanded, can be measured in situ.

### BACKGROUND OF THE INVENTION

I. Field of the Invention

The present invention relates to the sizing and imaging of cardiovascular defects generally and, more particularly, to producing images or forms that mimic the size and shape of defects and allow accurate sizing of implant devices. The defects treated are typically, but not limited to, openings or breaches in atrial or ventricular cardiac septum walls, so that remedial implant devices of the correct size and shape can be administered to occlude the defects. The invention specifically relates to a type of device and method which uses a three-dimensional open wire imaging structure which is flexible, easy to position, does not occlude blood flow or compromise a patient's hemodynamics and which may be expanded to detail the geometry of a defect being measured.

II. Related Art

Over the years, technology has developed such that many procedures can be accomplished using vascular catheters or guidewire systems rather than invasive surgery. This includes the implantation of devices and also the use of devices introduced over guidewires for discovering and sizing various types of defects and thereafter for introducing devices to treat the defects. Devices introduced by such techniques include stents to support vessel walls and occluders to occlude defects or close abnormal openings within the body. Additionally, balloon catheters have been used for sizing so that the standard occluder of the proper size is later deployed.

Memoried balloon-type devices are shown in U.S. Pat. Nos. 6,203,508 and 6,432,062 issued to Ren et al. In these references, the imaging balloon can be inflated to image the lesion of interest and the balloon thereafter deflated and withdrawn from the body. Upon re-inflation, the balloon resumes the memoried shape thereby providing a three-dimensional image of the geometry of the body lesion being measured. A further imaging balloon patent to Adams et al., U.S. Pat. No. 5,316,016, is directed to an imaging balloon catheter which illustrates a central area of reduced diameter or “waisted” area in the inflated imaging balloon.

While balloons have been used successfully for sizing defects, there are associated drawbacks. For example, when the balloon is inflated, it creates a temporary blockage that interferes with the hemodynamic performance of the circulatory system. Balloon devices also are quite slippery and have been found to be quite difficult to keep in position in membrane openings during the sizing procedure. It may also be difficult to control the pressure and thus the radial force exerted by the balloon as it is inflated which can lead to undersizing or oversizing of defects.

Open wire loop structures are also known, for example, for endocardial electrical mapping in heart chambers. One such device is illustrated and described in U.S. Pat. No. 6,014,579 to Pomeranz et al. These structures are heavily electroded and difficult to maneuver and control.

Despite previous progress, there remains a definite need in the art to provide an accurate imagining or sizing device for correctly diagnosing defects in the cardiovascular system which enables accurate, repeatable measurements for prostheses yet does not occlude blood flow during the imaging procedure or cause uncontrolled distortion of the structure being measured.

### SUMMARY OF THE INVENTION

By means of the present invention there is provided a variety of open wire sizing structures including wire mesh structures that can be used to determine the size and shape of a defect or abnormal opening in an accurate manner. The open wire structure concept allows blood to flow past the device at a virtually normal rate as it is being used to image and size a defect. The imaging structures are designed to be collapsed to a low profile in an elongated shape so that they may be introduced through the cardiovascular circulatory system and advanced to the vicinity of the defect. The devices may be self-guided, introduced over a guidewire and/or through a catheter lumen to the site of the defect.

The open wire sizing structures may be either of an operator actuated type or of a self-expanding type. In the case of operator actuated systems, an actuating member, preferably a wire attached to the distal portion of the imaging structure, may be displaced axially toward the proximal end to expand or deploy the open wire imaging structure to measure or size aspects of a defect sought to be repaired and displaced toward the distal end to collapse the structure. Self-expanding devices are made of materials that are memoried and can be heat set to remember a desired shape such as an oval or “balloon” shape. Constraints or wires are used to collapse the devices until deployed in situ and the collapsed state is re-established when a device is withdrawn and removed. When the device is released, it expands until it meets resistance such as from the edges of a defect being measured.

The open wire imaging structure itself may be any of several types of metallic structures including a braided or similar open woven wire mesh structure, a generally oval braided open wire mesh structure having a defined narrowed central “waist”, a plurality of radially distributed individual wire members connected between adjustably spaced proximal and distal end members or even a simple wire loop structure. The actuating or operating member, if present, is generally attached to the distal end of the open wire structure so that movement of the actuating member relative to a deployment guidewire, catheter, or the like, expands and collapses the open wire structure. The imaging structure might also be a non-metallic device constructed from a memoried polymer material so as to enable a return to the shape of the expanded form after having been collapsed and removed from the patient and thereby caused to resume the size and shape of the defect.

In accordance with the invention, however, the dimensions of the expanded sizing structures are preferably measured in situ using fluoroscopy. A device in accordance with the invention, for example, may be provided with markers or constructed of material which enables it to be visible under fluoroscopy during the procedure. A companion wire or parts of the device can be provided with marker bands or dimensional scales in any pattern to enhance fluoroscopic visibility and dimensional accuracy. Also, the device itself, without markers, may be made visible under fluoroscopy based on the diameter and density of wires making up the structure. In addition, other imaging techniques such as ultrasound may be used with the device.

The sizing structures of the invention are particularly useful in penetrating a defect in the form of an abnormal opening or breach in a membrane. The devices are expanded straddling the defect and form a waist that defines the shape and size of the opening so that a properly sized remedial occlusion device can be constructed. The radial force exerted by the device is designed to be able to very closely approximate that of the tissue surrounding the defect being measured (i.e., very little enlarging of the defect). The amount of radial stretch imparted on a defect can be closely controlled in some embodiments. The radial stretch is designed to mimic the radial stretch of the occlusion device that is going to be used to occlude the opening. By the sizing structures having the same radial stretch as the devices, the most appropriate occlusion devices can be selected.

A preferred structure is a braided wire mesh utilizing nitinol wire having an approximate diameter of 0.0015-0.008 inches formed as a mesh having approximately 4-144 wires. The device may be made in a variety of sizes regardless of type of construction.

### BRIEF DESCRIPTION OF THE DRAWINGS

In the drawings wherein like numerals denote like parts throughout the same:

FIGS. 1a-1c are schematic views of open wire imaging structures in accordance with the present invention which use an operator-controlled actuating member shown in expanded (1a) elongated, fully collapsed (1b) and deployed (1c) mounted on a guidewire;

FIGS. 2a-2f are schematic views illustrating self-expanding imaging structures in accordance with the invention;

FIGS. 3a-3c are photographic views showing open wire imaging structures in accordance with the invention of different sizes straddling a simulated opening in a membrane in several stages of expansion;

FIGS. 4a-4c illustrate schematically alternative embodiments of an open wire imaging structure in accordance with the invention; and

FIGS. 5a-5b illustrate possible associated measurement techniques that can be used in sizing and operating devices in accordance with the invention.

### DETAILED DESCRIPTION

The imaging structures of the invention are particularly suited to penetrating and sizing defects in the form of abnormal openings in bodily membranes particularly membrane walls such as heart septum membranes separating atrial or ventricular chambers so that an occluding repair device can be properly sized. It will be appreciated, however, that the imaging structure of the invention may also be used to image and size other types of defects including vascular stenoses. The embodiments of the detailed description which follows are offered as illustrations of the inventive concept and are not meant to be limiting in any manner.

As indicated, the expansion of the sizing structures can be controlled in one of several ways. One way is to use embodiments that expand on their own after being advanced into a defect and unsheathed or otherwise having the force holding them in a compressed state relieved. The sizing structure then expands to fill and size a defect. In other embodiments, an operator controlled actuating member can be used. The actuating or operating member is an axial element which extends through the length of the device and is operated from outside the body. The system may be calibrated so that the radial force exerted on the defect can be precisely controlled and the expanded size observed.

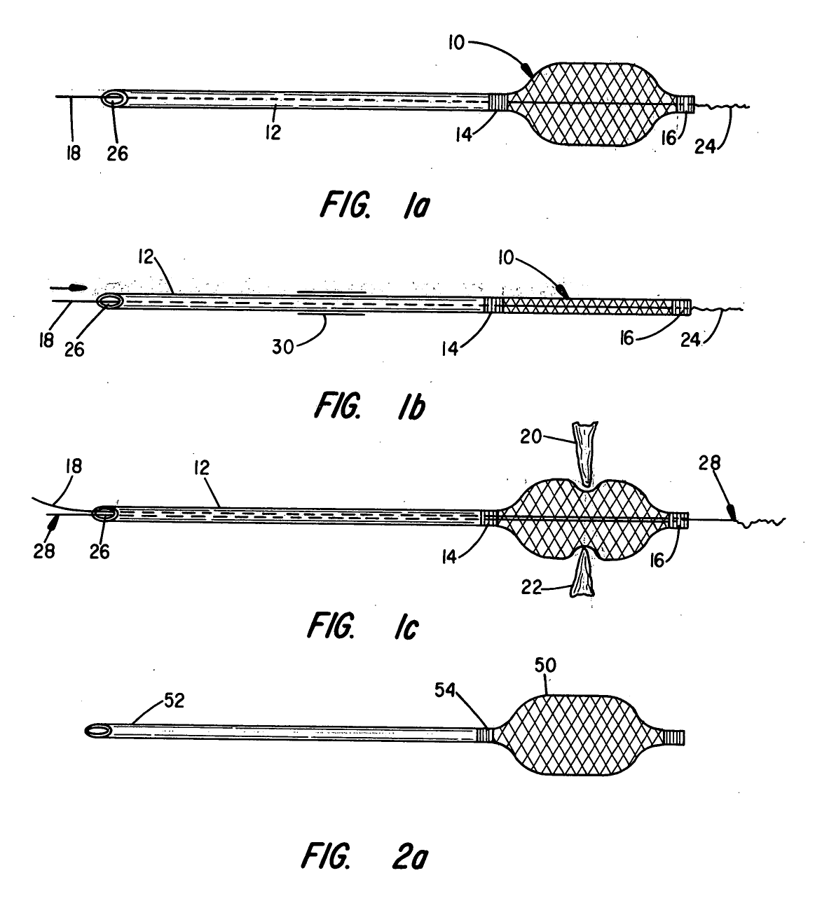

FIGS. 1a-1c depict an open wire imaging device structure including a generally oval or ovate shaped mesh structure 10 formed from braided woven wire. The structure 10 is attached to a deployment shaft 12 at a proximal end at 14 and has a free end 16. An operating or actuating wire is shown at 18 extending through the lumen of shaft 12 to a place of attachment at the distal end 16. The open wire structure 10 is shown expanded in an uninhibited manner in FIG. 1a, in the elongated or fully collapsed state suitable for deployment into the cardiovascular system of a patient in advancement to the location of a defect to be measured in FIG. 1b and as deployed in a tissue defect opening in tissue 20 in FIG. 1c. The basic image device structure of FIGS. 1a-1c is that of an embodiment that is usually an operator or user-controlled system in which axial movement of the actuation wire by the operator displaces the distal end 16 in relation to the proximal end 14 to cause elongation (collapse) or expansion of the structure 10. It should be noted, however, that the structure 10 may also be one that has been previously heat set and that upon release of the constraining force of the actuation wire 18, will self-expand and attempt to resume its heat set shape. As shown in FIG. 1c, the structure 10 is constrained from resuming or achieving the shape of FIG. 1a by the size of the tissue defect 20 thereby forming a defect sizing waist as at 22.

The FIGS. 1a-1c are also presented to illustrate several possible modes of deployment for the device into the cardiovascular system of a patient. The embodiment of FIG. 1a is shown with an optional atraumatic tip 24 such that the device may be used as a self-steering system when collapsed in the manner of FIG. 1b. As shown in the figures, the shaft 12 is preferably hollow and provided with a lumen 26 that extends through the length of the shaft to accommodate the actuation wire and so that the device can be advanced over a guidewire as at 28 in FIG. 1c. Also, the entire device may also be advanced through the lumen of a sheath or catheter as depicted by the fragment 30 in FIG. 1b.

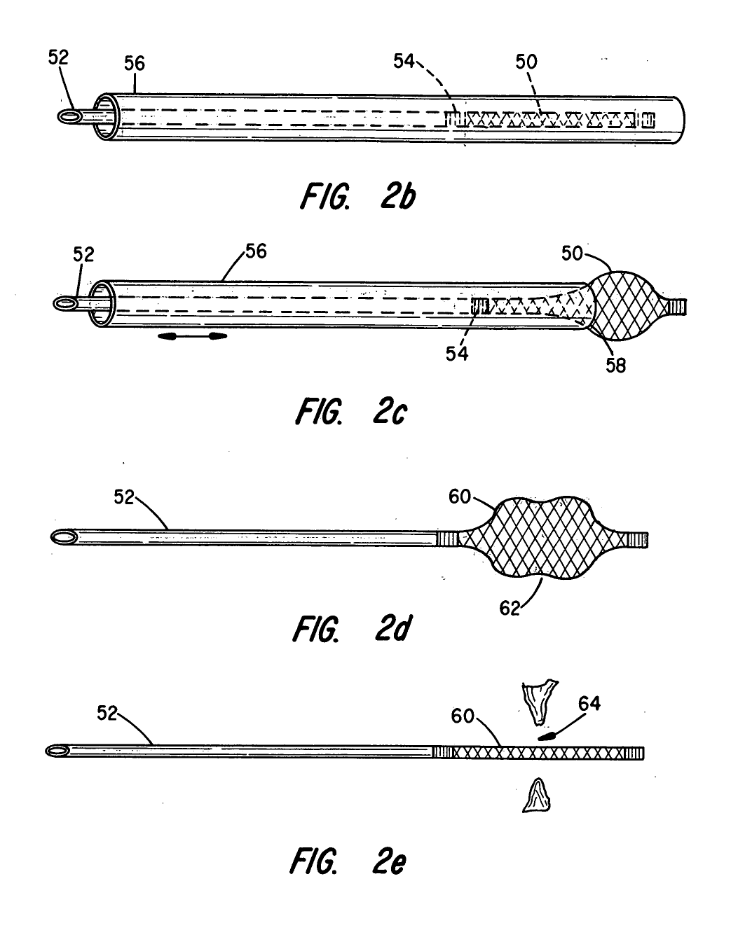

The FIGS. 2a-2f generally depict types of wire imaging or sizing structure embodiments similar to those in FIGS. 1a-1c but which are generally heat set, self-expanding sizing structures having been given an ovate or “balloon” shaped configuration. Thus, the device of FIG. 2a includes an open mesh sizing structure 50 shown in its heat set or self-expanded form fixed to a hollow flexible shaft 52 at the proximal end of the sizing structure at 54. FIG. 2b shows the sizing structure 50 constrained within an outer catheter or sheath 56 in position to be deployed with the catheter. FIG. 2c depicts the sizing structure 50 partially emerging from the distal end 58 of catheter 56 and either beginning to attempt to resume the shape depicted in FIG. 2a or, is in the process of being retracted into the sheath.

FIGS. 2d-2f show an alternate embodiment in which a self-expanding sizing structure 60 is provided with a slight pre-formed waist at 62 in its initial heat set shape. This configuration is of assistance in maintaining the location of the sizing structure within or straddling the defect during expansion. FIGS. 2e and 2f show one expansion process for a measuring device such as that shown in FIG. 2d with the device positioned in a defect 64 and ready to expand. FIG. 2f shows the sizing structure 60 fully expanded within defect 64 with the measurement 66 depicted as that which the sizing structure would measure as a diameter of the defect.

Of course, as previously indicated, the self-expanding or heat set sizing structures in accordance with the invention can also be constrained by using an actuating wire to elongate and then release the self-expanding sizing structure. In this manner, an actuating wire as at 18 in FIG. 1b could be used to elongate and collapse a self-expanding sizing structure for movement through the cardiovascular system and be locked in place in relation to the shaft to hold the device in the fully collapsed or elongated configuration as needed. When the device is located in situ, the “lock” can be released so that the sizing structure will attempt to return to its heat set form and thus be used to size a defect.

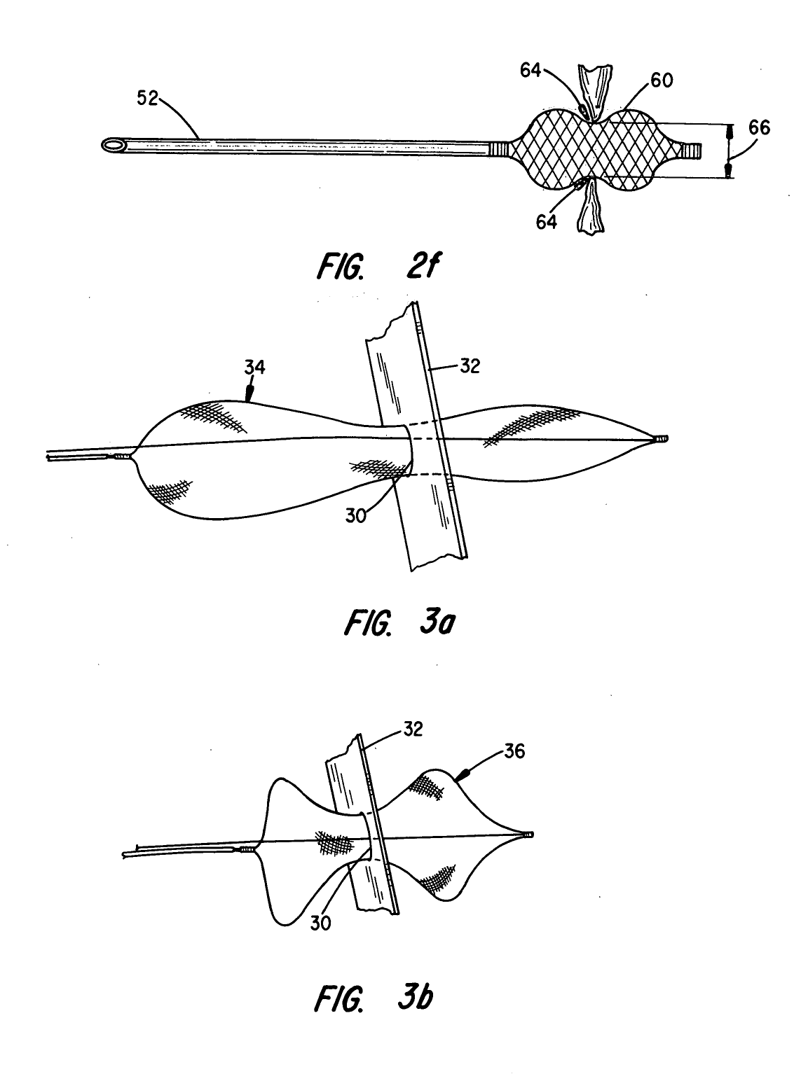

FIGS. 3a-3c depict open wire imaging or sizing structures similar to those of FIGS. 1a-1c positioned within a model defect 30 simulated in a silicon membrane 32 and expanded to fill the defect. The imaging or sizing structures shown are of three sizes and are identified by the reference characters 34, 36 and 38, respectively. As indicated above, the narrow sector in the expanded open wire structure forms the waist and corresponds to the size and shape of the defect opening as defined by the expanded woven wire shape.

The amount of radial force exerted by the expanded sizing structure is an important consideration leading to the selection of a proper size of occluding device (or stent in the case of a vascular measurement). Assuming the same mesh construction for all, the FIGS. 3a-3c further illustrate the relatively wide variation in radial force which can be applied by the sizing structure. The structure 34 in FIG. 3a remains generally elongated and is expanded a relatively small fraction of its potential and so exerts a relatively small amount of radial force in the defect. The structure 36 in FIG. 3b is expanded a greater relative amount and exerts a relatively larger radial force on the defect; and, finally, the structure 38 in FIG. 3c is expanded to a degree that causes the proximal portion to assume a truncated configuration at 40 representing the exertion of maximal force on the defect.

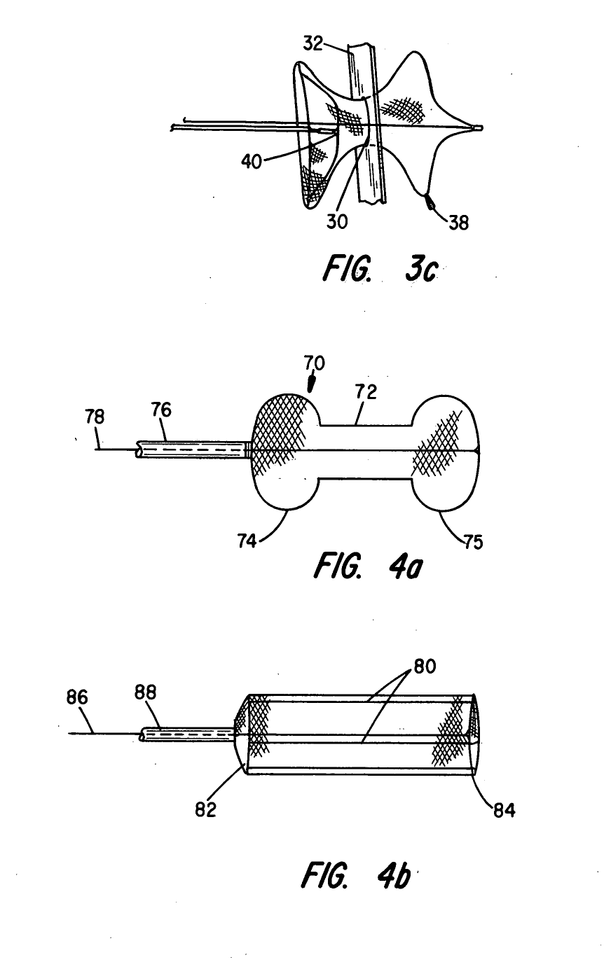

The FIGS. 4a-4c illustrate alternative embodiments of open wire structures in a fully expanded state. In this regard, FIG. 4a introduces a “dog-bone” shaped configuration 70 which includes an elongated waist portion 72 and two relatively larger end sections 74 and 75 with shaft 76 and actuating wire 78. The embodiment of FIG. 4b includes a small number of individual wires 80 connected between end configurations 82 and 84 in which relative motion of an actuating wire 86 and the shaft 88 cause the ends to converge or diverge and thereby expanding or collapsing the shape. FIG. 4c includes a pair of wire loops 90 and 92 which form the simplest construction of all for measuring defect size.

Of course, calibration measurements can be generated for a given size structure of known construction to relate expansion to defect size and shape to force in terms of relative displacement of guidewire and actuator wire or other defined relations.

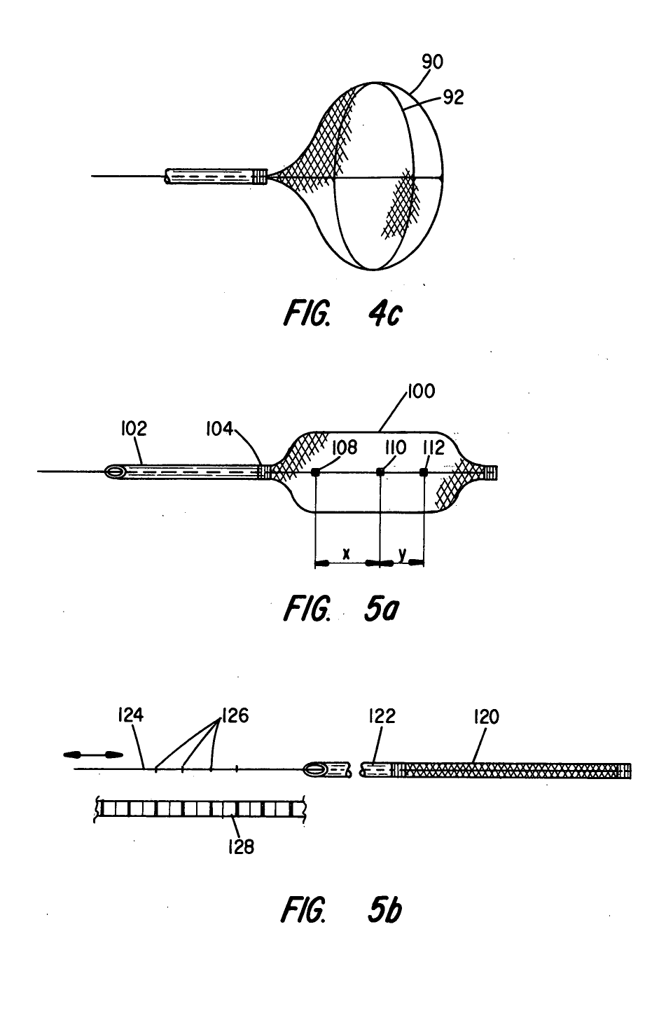

FIG. 5a depicts a sizing device 100 of the type previously described fixed to a shaft 102 at 104. An activation wire is shown at 106 and radiopaque markers are shown at 108, 110 and 112 toward the distal end of the activation wire 106. X and Y are known constant distances such that measurements made within the body can all be related accurately to these known distances. This is particularly useful with measurements made under magnified fluoroscopy (up to about 10×) which is normally used in such procedures. FIG. 5b shows a schematic representation including a collapsed sizing structure 120 attached to a shaft 122 (shown broken) and an actuating wire 124 which is provided with a series of calibration marks 126 situated alongside a measurement scale 128. This represents a calibrated system used to denote relative motion between a mark on the actuation wire and the scale corresponding to a given amount of sizing based radial expansion.

In operation, the three-dimensional open wire imaging structure is initially fully collapsed. If it is a steerable system, it can be introduced and advanced to the vicinity of a defect in the cardiovascular system. For other embodiments a catheter may be introduced into the vascular system of the patient and the imaging structure is advanced inside a catheter lumen or sheath to the vicinity of the defective of interest to be imaged or sized. Still other embodiments may be advanced over guidewires previously placed. The imaging structure is then expanded by using the actuating wire or releasing the device to self-expand to provide a measurement of the desired aspect of the defect of interest using the desired amount of force. Measurements are taken using fluoroscopy or other imaging techniques using sizing markers and the like to improve accuracy. The steps are then reversed and the sizing structure is collapsed and withdrawn from the patient.

An important aspect of the invention lies in the fact that the open wire nature of the sizing structures of the invention enables almost normal hemodynamics to continue in the patient. In addition, the wire structures, and particularly the mesh structures, provide added friction to the system which makes it easier to position the sizing structure in a defect and maintain its position during the measurement procedure.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A structure for imaging or sizing cardiovascular defects comprising:
(a) a three dimensional open wire structure adapted to be deployed into the cardiovascular system of a patient;
(b) an elongate device for advancing said imaging structure to the vicinity of a cardiovascular defect to be measured;
(c) a control system for controlling the operation of the open wire imaging structure in an imaging procedure;
(d) wherein said open wire imaging structure is selected from the group consisting of a generally oval braided open wire mesh structure, a generally oval braided open wire mesh structure having a defined central waist area, a plurality of radially distributed individual wire members connected between adjustably spaced proximal and distal end members, and a wire loop structure.

2. A device as in claim 1 wherein the wire mesh structure is a braided generally oval shape.

3. A device as in claim 1 wherein the wire mesh structure is woven.

4. A device as in claim 1 wherein said imaging structure is memoried so as to self-expand when released.

5. A device as in claim 1 further comprising aspects visible under fluoroscopy.

6. A device as in claim 4 wherein said imaging structure is a nitinol mesh.

7. A device as in claim 1 wherein said control system includes an actuating wire.

8. A device as in claim 1 wherein said defect is an abnormal opening in a membrane and wherein said imaging structure penetrates said opening and, upon expansion, forms a waist that defines the shape and size of the opening.

9. A device as in claim 4 wherein said defect is an abnormal opening in a membrane and wherein said imaging structure penetrates said opening in a membrane and, upon expansion, forms a waist that defines the shape and size of the opening.

10. A device as in claim 9 wherein said imaging structure is a nitinol mesh.

11. A device as in claim 1 wherein said control system includes an element for modulating the radial force exerted by said imaging structure.

12. A device as in claim 1 wherein said elongate device is a guidewire.

13. A device as in claim 7 wherein axial displacement of said actuating wire is calibrated.

14. A device as in claim 1 wherein said open wire structure further includes markers of known separation distance visible under fluoroscopy.

15. A device as in claim 1 wherein said elongate device is a catheter.

16. A device as in claim 1 that is a steerable system.

17. A device as in claim 1 wherein said open wire structure includes a polymeric material.

18. A method of imagery or sizing cardiovascular defects comprising steps of:
(a) providing a deployment device with a three-dimensional open wire imaging structure carried near the distal end thereof;
(b) introducing the deployment device into a patient and advancing the imaging structure to the vicinity of a defect of interest to be measured;
(c) expanding the imaging structure to provide a measurement of a desired aspect of the defect;
(d) determining the dimensions of the defect in situ;
(e) collapsing the imaging structure; and
(f) withdrawing the imaging structure from the patient.

19. A method as in claim 18 including the step of modulating radial expansion force in the imaging structure to control radial force exerted on the defect.

20. A method as in claim 18 including the step of measuring the imaging structure expansion in situ using fluoroscopy.

21. A method as in claim 18 including the step of measuring the imaging structure expansion in situ using ultrasound.
